(19) United States
(12) Patent Application Publication
(10) Pub. No.: US 2011/0276147 A1
(43) Pub. Date: Nov. 10, 2011

(54) Bone Graft Substitutes and Methods Thereof

(75) Inventors: Russell Cook, Costa Mesa, CA (US); Duraid Antone, Aliso Viejo, CA (US)

(73) Assignee: NUVASIVE, INC., San Diego, CA (US)

(21) Appl. No.: 13/187,340

(22) Filed: Jul. 20, 2011

Related U.S. Application Data
(63) Continuation of application No. 11/458,681, filed on Jul. 20, 2006.

(51) Int. Cl. A61F 2/28; A61B 19/00

(52) U.S. Cl. 623/23.51; 206/438

(57) ABSTRACT

An osteoinductive bone graft substitute composition that does not return to its original shape upon hydration or manipulation is disclosed, comprising, in combination, about 86-89% by weight of a calcium phosphate particulate mineral component and about 11-14% by weight of a purified fibrillar collagen, the mineral component including about 20% to about 60% by weight of hydroxyapatite and about 60% to about 20% by weight of tricalcium phosphate. A package configured to store a bone graft composition is disclosed, comprising an inner sterile polymeric V-shaped pouch located in an outer sterile polymeric V-shaped pouch. Methods for repairing a bone defect in a patient are disclosed using the osteoinductive bone graft substitute composition.

### FIELD OF THE INVENTION

This invention relates generally to bone graft substitutes and, more specifically, to improved collagen and ceramic-based bone graft substitutes, and methods of making and administering collagen and ceramic-based bone graft substitutes.

### BACKGROUND OF THE INVENTION

Numerous types of bone graft substitutes are currently available. Typically, such bone graft substitutes comprise composites of one or more types of materials usually built on a base material.

By way of example, according to one classification scheme allograft-based bone graft substitutes comprise allograft bone used alone or in combination with other materials.

The term “allograft” means a tissue graft between genetically different organisms.

Furthermore, factor-based bone graft substitutes comprise natural and/or recombinant growth factors combined with other materials such as transforming growth factor-beta [TGF-beta], platelet-derived growth factor (PDGF), fibroblast growth factor (FGF), and bone morphogenetic protein (BMP), hyaluronic acid, or a thermoplastic lactide. The classification scheme described above also encompasses bone graft substitutes which may be characterized as a cell-based bone graft substitute using cells to generate new tissue alone or seeded onto a support matrix. Polymer-based bone graft substitutes are yet another member of the classification scheme and include both degradable and non-degradable polymers used alone and in combination with other materials. One example of a polymer based bone graft substitute HEALOS® Bone Graft Material (DePuy Orthopaedics, Inc., U.S.A.) is a polymer ceramic composite consisting of collagen fibers coated with hydroxyapatite which has been indicated for spinal fusions.

Other examples of polymer-based bone graft substitutes available from Orthovita, Inc., U.S.A. include Foam®, a collagen and ceramic composite, Vitoss®, a tricalcium phosphate material, Cortoss®, an injectable resin-based product with applications for load-bearing sites and Rhakoss®, a resin composite available as a solid product in various forms for spinal applications.

A majority of bone graft substitutes are ceramic-based and include calcium phosphate, calcium sulfate, and bioglass used alone or in combination with other materials. Calcium phosphates are a primary inorganic component of bone in the form of calcium hydroxyapatite or hydroxylapatite Ca₁₀(PO₄)₆(OH)₂ (abbreviated as HA). The term calcium phosphate encompasses a family of minerals containing calcium ions (Ca²⁺) together with orthophosphates (PO₄³⁻), metaphosphates or pyrophosphates (P₂O₇⁴⁻) and occasionally hydrogen or hydroxide ions. Tricalcium phosphate occurs in alpha and beta phases and the beta phase is the most common form of “calcium phosphate”. Tricalcium phosphate, synthetic hydroxyapatite, and coralline hydroxyapatite are available in pastes, putties, solid matrices, and granules. Additionally it is known that calcium phosphates are osteoconductive, osteointegrative and may be osteoinductive. During synthesis, calcium phosphates often require high temperatures for scaffold formation and when used alone are brittle. Consequently, calcium phosphates are formulated with other materials to create composites with improved mechanical properties.

Calcium sulfate is biocompatible, bioactive, and resorbable after 30-60 days. However, calcium sulfate undergoes significant loss of mechanical properties when degraded, making use of this ingredient in bone graft substitute formulations questionable in load-bearing structural applications.

Bioactive glass (also termed “bioglass”) is a biologically active silicate-based glass having a high modulus with brittle properties resulting in limitations of use in applications. However, once again formulations that overcome some of these limitations provide some useful bioglass-based bone graft substitutes.

Additionally, most ceramic-based bone graft substitutes do not include a collagen component. Although there are a number of different types of collagen, Type I collagens are the most abundant collagens found in humans. Collagen is a long fibrous structural protein found in animal tissue and is often derived from cows when used medically. Collagen is tough and inextensible, with great tensile strength, and is the main component of cartilage, ligaments and tendons, and the main protein component of bone and teeth. Therefore, a formulation including calcium phosphates and collagen may provide a synergistic combination of structural materials useful in bone graft substitute applications.

Examples of ceramic-based bone graft substitutes include OsteoGraf® (Dentsply International, U.S.A.), Norian® SRS (Norian Corp., U.S.A., a subsidiary of Synthes-Stratec, Inc.), Collagraft® and Neugraft® (Angiotech Pharmaceuticals Inc., Canada, and Zimmer, Inc., Collagen Corp. and Neucoll Corp., U.S.A.), COPIOS™ (Zimmer, Inc.) and many other similar substitutes. Many of these ceramic-based bone graft substitutes have been disclosed in multiple patents associated with their composition, production methods, and their combination with devices of various kinds and the like. These patents disclose that the bone graft substitutes currently used include collagen obtained in various ways.

For example, Wallace, U.S. Pat. No. 4,789,663, discloses collagen derived from demineralized bone. In a second example, Yamamoto, U.S. Pat. No. 6,764,517 discloses: “a composition comprising an effective amount of a porous, biodegradable three-dimensionally fixed matrix having shape memory comprising insoluble mineralized biopolymer fibers, etc.” In a third example, Piez, U.S. Pat. Nos. 4,774,227, 4,795,467 and 5,425,770 discloses the use of atelopeptide reconstituted fibrillar collagen. These examples are merely illustrative of the plethora of patents issued relating to bone graft substitutes. U.S. Pat. No. 6,180,606, to Chen, discloses the use of a porous or semi-porous matrix, which may be collagen, with an osteoinductive factor and a growth factor that may be a calcium compound, which may be the same as the osteoinductive factor, and which may comprise demineralized bone particles, in which the ratio of collagen to demineralized bone particles is 10% to 90% collagen and 90% to 10% demineralized bone particles. Chen does not disclose specific ratios and materials that may optimally create a bone graft without shape memory. Rhee, U.S. Pat. No. 5,264,214, requires chemical conjugation of collagen to a synthetic hydrophilic polymer.

Shape memory (the reformation of a bone graft substitute into its original shape when moisture is added) was once considered desirable but may create problems in that the substitute will, after it has been inserted and the surgical wound closed, return to its original shape causing it to separate from the bone cavity producing a poor host to graft interface.

In addition, a number of patients are allergic to collagen. In efforts to solve that problem, several inventors, including Piez, U.S. Pat. Nos. 4,774,227, 4,795 and 5,425,770 developed methods to remove the regions at each end of collagen which do not have the triplet glycine sequence, and thus do not form helices that are thought to be responsible for the immunogenicity associated with most collagen preparations, to produce “atelopeptide” collagen. This may be accomplished by digestion with proteolytic enzymes, such as trypsin and pepsin. The non-helical telopeptide regions are also responsible for natively occurring cross-linking, and atelopeptide collagen must be cross-linked artificially if cross-linking is desired.

However, once digested according to the method of Piez and others, the atelopeptide collagen has been disassembled into individual triple helical molecules and must then be reconstituted to regroup into its fibrillar form. In this form, the fibrils consist of long, thin collagen molecules staggered relative to one another by multiples of about one-fourth their length. This results in a banded structure which can be further aggregated into fibers. In all cases, bone graft substitutes described in the prior art require fairly complicated formulations with accompanying complicated methods of preparing such formulations. This appears to be because each formulation is supposed to address some deficiency of prior art formulations and of course provide other perceived benefits. The current disclosure provides a different approach for formulating and producing bone graft substitutes.

### SUMMARY OF THE INVENTION

The current disclosure provides an improved bone graft substitute comprising one or more compositions that differ from any of the prior art. In an exemplary embodiment a bone graft substitute composition comprises a purified fibrillar collagen and a partially resorbable hydroxyapatite/tricalcium phosphate (HA/ TCP) ceramic in a range of proportions of 11-14% by weight of resorbable purified fibrillar collagen and the HA/TCP component including between 20-60% by weight of HA that does not have shape memory, and does not require reconstitution of the collagen to reduce allergenicity. Allergenicity is reduced by the purification process of Nimmi, U.S. Pat. No. 5,374,539, whose disclosure is incorporated herein by reference, and provides a method that allows for enzymes to reach such areas of the fibril and remove these non-helical extensions without dissociating the fibers into individual molecules and causing the fibrils to disassemble. The compositions of this invention are purified in this manner so that the complicated process of reconstitution is avoided. The immunogenicity is controlled by the purification process and the relatively low levels of collagen (11-14% by weight) as disclosed herein. This disclosure provides an improved bone graft substitute composition which avoids the need for reconstitution of collagen as disclosed by Wallace, U.S. Pat. No. 4,789,663, and others, does not have shape memory, and provides an effective means of filling a defect in bone.

In an aspect of this disclosure, the bone graft substitute composition comprises highly purified type I collagen and a ceramic, which may be hydroxyapatite/tricalcium phosphate granules. It functions as an osteogenic stimulus to which the patient's bone marrow is added prior to implantation. The bone graft mimics the composition of natural bone and is biocompatible. The composition provides an osteoconductive environment for new bone formation. When coated with autogenous bone marrow, the osteoinductive and osteogenic properties of the composition enable it to be used as a substitute for autogenous bone graft, thus eliminating the need to subject the patient to the potential attendant morbidity as well as the harvesting-related complications and pain associated with a second surgery.

In another aspect of this disclosure the bone graft substitute composition is osteoconductive, osteointegrative and may be osteoinductive, yet requires only purified fibrillar collagen and a ceramic, specifically hydroxyapatite/tricalcium phosphate without the need for other materials such as transforming growth factor-beta [TGF-beta], platelet-derived growth factor (PDGF), fibroblast growth factor (FGF), and bone morphogenetic protein (BMP), hyalin or thermoplastic lactide.

Advantageously, the compositions comprise relatively inexpensive components and may be readily prepared thereby providing economic benefit to users of the compositions. In one aspect the compositions may comprise highly purified Type I collagen and hydroxyapatite/tricalcium phosphate (HA/TCP) granules, wherein a patient's bone marrow is added to the composition prior to implantation. When coated with autogenous bone marrow, the osteoinductive and osteogenic properties of the compositions may enable use as substitutes for autogenous bone grafts.

Beneficially, the compositions eliminate the need to subject the patient to the potential attendant morbidity as well as the harvesting-related complications and pain associated with a second surgery. Without limiting the disclosure, it is understood that the compositions may comprise other sources of Type I collagen. In yet another aspect the HA/TCP components of the compositions may comprise a biphasic mixture and may be formed by a sintering process into irregularly-shaped granules. The purified collagen and HA/TCP composite may serve as a matrix for an osteogenesis process to occur. Advantageously, in situ, the collagen and beta-tricalcium phosphate components of the improved bone graft substitute compositions may be resorbed and replaced by new bone similar to the resorption and remodeling observed with an autogenous bone graft.

In accordance with an embodiment of this invention, an osteoinductive bone graft substitute composition is disclosed. The composition comprises, in combination about 86% to about 89% by weight of a calcium phosphate particulate mineral component and about 11% to about 14% by weight of a purified fibrillar collagen, the mineral component including about 20% to about 60% by weight of hydroxyapatite and about 80% to about 40% by weight of tricalcium phosphate, wherein the composition does not return to its original shape upon hydration or manipulation.

Furthermore, the tricalcium phosphate substantially comprises beta tricalcium phosphate and the collagen substantially comprises Type I non-reconstituted bovine collagen.

In an aspect the composition comprises about 87.5% by weight of calcium phosphate particulate mineral component and about 12.5% by weight of purified fibrillar collagen, wherein the mineral component comprises about 30% to about 50% by weight hydroxyapatite and about 70% to about 50% by weight tricalcium phosphate. In another aspect, the composition comprises about 87.5% by weight calcium phosphate particulate mineral component and about 12.5% by weight purified fibrillar collagen, wherein the mineral component comprises about 50% by weight hydroxyapatite and about 50% by weight tricalcium phosphate.

Furthermore, the calcium phosphate particulate mineral component comprise irregularly-shaped granules having a diameter of about 0.5 mm to about 1 mm.

The composition may be configured as at least one of a strip and a block and have a structure including one or more interconnected pores, wherein the one or more pores have an average pore size of about 100 mm to about 800 mm. The at least one of a strip and a block have a thickness of about 1 mm to about 5 mm and in an aspect have a thickness of about 3 mm. The at least one of a strip and a block have a width of about 3 mm to about 100 mm and a length of about 3 mm to about 100 mm. In another aspect the composition may be configured into strips only, and in one aspect the strips may be 15 mm wide and 45 mm long.

In yet another aspect the composition may further comprise an effective amount of one or more compounds selected from the group consisting of autogenous bone marrow, transforming growth factor-beta (TGF-beta), platelet-derived growth factor (PDGF), fibroblast growth factor (FGF), bone morphogenetic protein (BMP), hyaluronic acid, thermoplastic lactides and antibiotics, the one or more compounds configured to promote regrowth of bone.

In accordance with another embodiment of this invention, an osteoinductive bone graft substitute composition comprises, in combination about 86-89% by weight of a ceramic component and about 11-14% by weight of a purified fibrillar collagen, wherein the composition will not return to its original shape upon hydration or manipulation.

In accordance with yet another embodiment of this invention, a package configured to store a bone graft composition is disclosed. The package comprises, in combination at least one of a strip and a block of the bone graft composition including about 86-89% by weight of a calcium phosphate particulate mineral component and about 11-14% by weight of purified fibrillar collagen, the mineral component including about 20% to about 60% by weight hydroxyapatite and about 60% to about 20% by weight beta-tricalcium phosphate, wherein the composition does not return to its original shape upon hydration or manipulation; and the package comprises an inner sterile polymeric V-shaped pouch located in an outer sterile polymeric V-shaped pouch.

In accordance with still another embodiment of the invention a method for repairing a bone defect in a patient is disclosed. At least one of a strip and a block of an osteoconductive bone graft composition are provided and comprise about 86-89% by weight of a calcium phosphate particulate mineral component and about 11-14% by weight of purified fibrillar collagen substantially comprising Type I collagen, the mineral component including about 20% to about 60% by weight hydroxyapatite and about 60% to about 20% by weight beta-tricalcium phosphate, wherein the composition does not return to its original shape upon hydration or manipulation. Furthermore, the patient is placed in an aseptic operating room, a wound is opened to access the bone defect, the bone defect is filled with at least one of the strip and the block, and the wound is closed. In an aspect bone defect has a volume of no greater than about 30 ml.

In further steps of the method the wound associated with the bone defect is debrided and managed prior to filling the bone defect, wherein periosteal stripping is minimized and a contaminated portion of the wound is treated with at least one prophylactic antibiotic. In yet a further step, autogenous bone marrow is collected from at least one of an iliac crest and a fracture site of an uncontaminated wound while avoiding blood collection with the bone marrow. At least one of the strip and the block are sized to fit into the bone defect and transferred to a sterile tray. Furthermore, a sterile saline solution is added to the sterile tray and at least one of the strip and the block are hydrated for a period of about one to three minutes. Another sterile tray including the bone marrow is provided and the at least one of the strip and the block are transferred to the another sterile tray. In further steps, at least a portion of the surface of the at least one of the strip and the block are coated with the bone marrow and at least one bone graft composition is placed into the bone defect so that molding is minimized, wherein if molding is required bone marrow cells of the bone marrow are substantially undamaged. In another aspect of this embodiment about 5 ml of bone marrow is coated onto every 15 mm by 45 mm strip of the composition.

In other aspect of the method, an internal fixation device may be inserted prior to selecting at least one strip sized to fit into the bone defect. Furthermore, an external fixation device may be inserted after closure of the wound. The bone defect may be surgically created or result from bone trauma.

The foregoing and other articles, features, and advantages of the invention will be apparent from the following more detailed description of the preferred embodiments of the invention. The various features may be utilized or claimed alone or in any combination.

### DESCRIPTION OF THE INVENTION

In the following description, numerous specific details are set forth in order to provide a more thorough description of the present invention. It will be apparent, however, to one skilled in the art, that the present invention may be practiced without these specific details. In other instances, well-known features have not been described in detail so as not to obscure the invention.

In the Summary above, the Description of the Invention, and the Claims and Abstract below, reference may be made to particular features (including method steps) of the invention. It is to be understood that this disclosure includes possible combinations of such particular features. For example, where a particular feature is disclosed in the context of a particular aspect or embodiment of the invention, or a particular claim, that feature may also be used, to the extent possible, in combination with and/or in the context of other particular aspects and embodiments of the invention, and in the invention generally.

The term “comprises” and grammatical equivalents thereof are used herein to mean that other components, ingredients, steps etc. are optionally present. For example, an article “comprising” (or “which comprises”) components A, B and C can consist of (i.e. contain only) components A, B and C, or can contain not only components A, B and C but also one or more other components. Where reference is made herein to a method comprising two or more defined steps, the defined steps can be carried out in any order or simultaneously (except where the context excludes that possibility), and the method can include one or more other steps which are carried out before any of the defined steps, between two of the defined steps, or after all the defined steps (except where the context excludes that possibility).

The term “at least” followed by a number or the indefinite article “a” (meaning “one”) is used herein to denote the start of a range beginning with that number (which may be a range having an upper limit or no upper limit, depending on the variable being defined). For example “at least one” or “at least a” means 1 or more than 1. The term “at most” followed by a number is used herein to denote the end of a range ending with that number (which may be a range having 1 or 0 as its lower limit or a range having no lower limit, depending upon the variable being defined). For example, “at most 4” means 4 or less than 4, and “at most 40%” means 40% or less than 40%. If, in this disclosure, a range is given as “(a first number) to (a second number)” or “(a first number)-(a second number)”, this means a range whose lower limit is the first number and whose upper limit is the second number. For example, 0-10 mm means a range whose lower limit is 0 mm, and whose upper limit is 10 mm.

The term “or” is used herein as a conjunction used to link alternatives in a series of alternatives. The term “and/or” is used herein as a conjunction meaning that either or both of two options may be valid.

In an exemplary embodiment an osteoinductive bone graft substitute composition for use in bony voids or gaps that are not intrinsic to the stability of the bony structure is disclosed. In one embodiment, the bone graft substitute composition may be gently packed into bony voids or gaps of the skeletal system in the extremities, spine, pelvis, etc. These defects may be surgically created osseous defects or osseous defects created from traumatic injury to the bone and should have a volume of 30 ml or less. The disclosed composition provides a bone void filler that resorbs and is replaced by the growth of new bone during the healing process. The bone graft may be mixed with autogenous bone marrow prior to use at a physician's discretion. In weight bearing situations, the composition may be used in conjunction with internal or external fixation devices.

According to an embodiment of this disclosure the osteoinductive bone graft substitute composition does not return to its original shape upon hydration or manipulation and comprises a mixture of about 86-89% by weight ceramic mineral component. In an embodiment, the ceramic mineral component comprises calcium phosphate, specifically, a mixture of about 20% to about 60% by weight of hydroxyapatite and about 60% to about 20% by weight of tricalcium phosphate, in admixture with about 11-14% by weight of purified fibrillar collagen. In an exemplary embodiment, the tricalcium phosphate may substantially comprise beta tricalcium phosphate and the collagen may substantially comprise Type I bovine collagen. In yet another exemplary embodiment, the composition may comprise about 87.5% by weight of calcium phosphate particulate mineral component and about 12.5% by weight of purified fibrillar collagen and the mineral component described above may comprise about 30% to about 50% by weight of hydroxyapatite and about 70% to about 50% by weight of tricalcium phosphate. In an exemplary embodiment the mineral component may comprise about 60% by weight of hydroxyapatite and about 40% by weight of tricalcium phosphate.

In an embodiment, the composition may optionally include autogenous bone marrow, transforming growth factor-beta [TGF-beta], platelet-derived growth factor (PDGF), fibroblast growth factor (FGF), and bone morphogenetic protein (BMP), hyaluronic acid, a thermoplastic lactide, an antibiotic and the like.

In another embodiment, the composition may be prepared as prefabricated strips and blocks having a thickness of about 1 mm to about 5 mm. In an exemplary embodiment the thickness may be about 3 mm. The strips may be prepared at any width and length, an in an embodiment may be between 3 mm and 100 mm wide and 3 mm and 100 mm long. When sized as 15 mm wide and 45 mm long the strips are particularly useful in repairing bone defects. The composition is suitable for any defect in the bone whether created by surgery or traumatically induced.

When the collagen source is a bovine fibrillar collagen component it has been determined in vitro and in vivo that this component is biocompatible and has low immunogenicity, making it a suitable material for providing a scaffold around which new bone can grow. Hydroxyapatite (HA) is radiopaque and highly biocompatible. HA is a polycrystalline substance with a stoichiometry similar to bone mineral and is minimally resorbed as bone grows into the scaffold. The porous β-TCP ceramic has a stoichiometry similar to amorphous biologic precursors to bone. In addition, it is biodegradable and its biodegradation products can be reconstituted by the body to form new bone mineral, allowing for bone deposition to occur. The porous HA/β-TCP ceramic has been shown to possess an osteoconductive property for filling bone defects and it can evoke a biologic response similar to that of bone.

In an aspect of the purified fibrillar collagen component described herein, the component has a high content of purified bovine tendon Type I collagen and is available from Maxigen Biotech, Inc., Taiwan as well as other U.S. sources.

As with other products containing collagen, the bone graft substitute composition of this disclosure may be unsuitable for use in patients with a history of severe allergies manifested by a history of anaphylaxis and known allergies to bovine collagen, patients known to be undergoing desensitization injections to meat products, as these injections can contain bovine collagen, children and pregnant women, operative sites with inflammatory bone disease such as osteomyelitis, fractures of the epiphyseal plate, in sites with severe vascular or neurological impairment proximal to the graft site, patients with a metabolic or systemic bone disorder, or in contaminated wounds with existing acute or chronic infections.

The mineral component of the above described compositions may comprise a biphasic mixture of substantially about 80% to about 40% by weight of β-tricalcium phosphate. In other aspects the β-tricalcium phosphate content may comprise about 50% to about 70% by weight of the mineral component or optionally 50% by weight of the mineral component with the balance comprising hydroxyapatite. In an exemplary embodiment the HA/β-TCP particles of the mineral composition are formed by a sintering process into irregularly-shaped granules of about 0.5-1 mm in diameter. The HA /β-TCP component is available from Berkeley Advanced Biomaterials as Bi-Ostetic™ Synthetic Bone Graft from Orthovita, Inc., of Malvern, Pa.

Further characteristics of the one or more compositions include a density of about 0.573 g/cm³, as determined from weights and dimensions of the compositions formed into appropriate shapes. Furthermore, the compositions have a porosity of about 50-75%, with an interconnected pore structure and an average pore size of about 100-800 micrometers. The compositions are radiopaque and may be presterilized for single use.

In an embodiment, the collagen to ceramic ratio comprises about 11% to 14% by weight of collagen to about 86% to about 89% by weight of ceramic. In a further exemplary embodiment, the collagen to ceramic ratio is about 12.5% to about 87.5%.

Exemplary methods for effecting osteoinductive repair of bone defects will now described with reference to the various embodiments of the bone graft substitute compositions (see description above).

### Exemplary Method for Effecting an Osteoinductive Bone Repair

An embodiment of a method for repairing a bone defect in a mammal using at least one embodiment of an osteoconductive bone graft substitute composition (see description above) a practitioner may comprise a number of steps. It is understood that the order of the steps may be changed without limiting the disclosure.

In one step the patient may be placed in an operating room under aseptic conditions following standard procedures for bone grafting with fixation as is understood in the art. In a further step a bone defect wound may be debrided and managed while exercising care to minimize periosteal stripping. Of course, if the bone defect is unexposed a wound may need to be opened. In yet another step one or more contaminated wounds may be treated with appropriate prophylactic antibiotics and the like.

During the method, bone marrow from the iliac crest or the fracture site of an uncontaminated wound may be collected while exercising care to avoid collecting blood instead of or admixed with the bone marrow; and appropriately shaped, sized, and number of pieces of the bone graft substitute composition (see description above) may be selected to fit the bone defect. The bone graft composition may be transferred to a sterile tray and sterile saline may be added to the tray, wherein the composition may be hydrate for about one to three minutes. Furthermore, the composition may be transferred to another sterile tray containing bone marrow and at least a portion if not all surfaces of the composition may be coated with the previously collected bone marrow. In one aspect, about 15 mm by about 45 mm strips about 3 mm thick are used, and about 5 ml of bone marrow is spread over each. The composition may be placed into the bone defect in such a way as to minimize the need to mold the composition. Significantly, since the composition has no shape memory the bone defect may be more readily filled completely. If molding is required care should be exercised to avoid crushing the composition or damaging the marrow cells. In an exemplary embodiment the bone defect is filled as completely as possible and the wound accessing the bone defect may be closed using standard operating techniques as understood in the art. p Alternatively, the method may be performed using the composition configured as prefabricated strips or blocks. Optionally, if the defect is in a weight bearing bone an internal fixation device may be inserted prior to the selection and insertion of the composition, whether as prefabricated strips or other solid pieces, or an external fixation device may be applied after the wound has been closed. p Without limiting the disclosure, the following describes various studies of the compositions disclosed herein when used in vivo.

### EXAMPLE 1

### Immunogenicity Study

### Materials:

One or more test strips comprising the compositions as described above was provided and extracted to liquid form at 4 gm/20 ml. Sodium Chloride, 0.9% (normal saline) and Freunds Complete Adjuvant (FCA) were obtained commercially. 5 ml of each, normal saline and the test extract were mixed with 5 ml of FCA.

### Animals:

Fifteen male and female Hartley guinea pigs at least 21 days old are obtained.

### Study Design:

Animals in both groups receive 3 injections, the test extract and FCA, normal saline, and normal saline and FCA on Day 1 in the shoulder area of the animal. On Day 6, 10% sodium laurel sulfate is massaged into the area of the injections. On Day 7, 2×4 cm patches soaked in normal saline are applied to the injection area of the control animals, and similar patches soaked in the test extract are applied to the test group animals and all patches are removed on Day 9. On Day 21, 2×2 patches soaked in normal saline or a control animal is applied to the left flank of each appropriate animal.

### Evaluation:

Statistical analysis is performed for the degree of sensitization as determined by skin erythema and edema at 24, 48 and 72 hours.

Neither the test animals nor the control animals show signs of erythema or edema where the patches were applied. According to normal statistical methods as applied in the art, this constitutes a Grade I or weak sensitizer response, and no differences are observed between the test and control animals.

### EXAMPLE2

Animal Study 2 Bone Graft in New Zealand white rabbits

### Materials:

One or more test strips comprising the compositions as described above is provided.

### Animals:

The experimental animals are New Zealand white rabbits, weighing about 2.8 to 3.5 kg. The animals are evaluated radiographically before operation to verify the absence of osseous abnormalities.

A total of 30 rabbits are randomly divided into six groups as follows: 5 animals each are sacrificed at one month, two months, three months, six months, nine months, and one year after surgery.

Surgical procedure

Surgery is performed following standard an aseptic technique under general anesthesia as is understood in the art. The bone healing model consists of a unicortical cylindrical bone defect in one distal femur of the rabbit. Following ablation, one or more strips comprising the compositions described herein are introduced into the bone defect. The lateral cortex is covered back to the created defect to contain the graft materials within the bone. The wound is then closed in layers. The animals are monitored postoperatively until they are able to walk around, and then returned to their cages. Antibiotic (cefacin 100 gm per dose) is administered intramuscularly, one shot before and after surgery separately.

Postoperative care

The animals are monitored daily for their activity and for any undue complications. Body weights are measured every two weeks until euthanasia. Euthanasia solution (sodium pentobarbital, 60 mg/kg) is used at 1, 2, 3, 6, 9 months, and one year after surgery. The distal femurs of the rabbits are harvested, and the medial and lateral condyles are bisected. Medial condyles are processed for decalcified histological analyses and lateral condyles are processed for undecalcified histomorphometric analyses.

### Evaluation

Anteroposterior and lateral radiographs of each animal's femur is made immediately after surgery, and at 1 month, 2 months, 3 months, 6 months, 9 months, and 1 year after surgery before they are sacrificed for further study. Magnetic Resonance Imaging (MRI) of the distal femur is performed during the same time sequence.

After sacrifice, the bones are subject to mechanical testing by burring after which the peak compression load of the defect is considered by manual palpation, the peak compression load of the cancellous-graft composite being assessed with a servohydraulic testing machine (Instron 5544, Instron Corporation, USA).

Histological, histomorphetric studies with radiography are performed. As new bone formed, the appearance of the graft materials became more radio-opaque. A distinct radiolucent zone at the interface between the graft materials and the host bone is visible on the immediate postoperative radiographs. The absence of this radiolucent zone is considered to be an indication of union between the graft materials and the surrounding host bone.

Light microscopy, combined with histomorphometric evaluation of the observed changes, is used to describe the healing characteristics of the bone defects and the reaction to the implanted materials. Generally, little inflammation is noted. Mild foreign body reaction is noted. The strips of the compositions described herein formed lumps that are enveloped by a thin fibrous sheath separating the granules from each other. A mild foreign body reaction surrounding the material lump is noted.

At one month after implantation, portions of strip granules of the composition described herein are found to be enveloped by a thin fibrous sheath separating the granules from each other. A mild foreign body reaction in the vicinity of the material is noted. There appeared to be a centripetal osteoconductive process emerging from the interface between graft material and host bone. In the vicinity of implanted materials, the mesenchymal cells proliferated and produced extracellular matrix. The cells start to differentiate into osteoblasts and formed immature new bone trabeculae. This bone tissue shows the structural characteristics of newly formed bone, i.e. a homogenous matrix without lamellation. The central region of the defect is filled with implanted materials.

By 2 months, histological examination shows that bone defect is filled with more newly formed bone. Bone formation appears to arise from the periphery between the host bone and the implanted materials towards the center of the defect. New bone already formed bridges between one another as well as between newly formed bones and the host bone.

By three months, new bone has formed bridges between a substantial fraction of the graft materials and has reached the center of the defect for both graft materials. Higher magnification shows that newly formed bone contained either woven or lamellar new bone and that the bone is in direct contact with the implanted materials. There is uniform and substantial new bone formation throughout the implanted materials with time. Resorption, or dissolution, of the graft materials is evident by this time period and appeared to be associated with bone formation in the resorption areas.

Bone growth continues to increase in density between 3 and 6 months. Thick, new trabecular bone completely encases the graft material. The trabeculae formed from defect filled with one or more strips of the compositions described herein were few, but thick and interconnected to the surrounding cortex and trabecular bones.

There is no evidence of obvious immune or inflammatory responses to the materials used in this study. Slight foreign body reaction for the strips is noted. Each strip allows new bone formation within three months after implantation, indicating its potential for use as a scaffold for in vivo bone growth. The strips are slowly resorbed with materials retained in the defect site for more than one year after implantation. Consequently, the repaired tissue comprises a composite of bone reinforced by the alloplastic materials, rather than pure bone.

While the invention has been particularly shown and described with reference to preferred embodiments thereof, it will be understood by those skilled in the art that the foregoing and other changes in form and details may be made therein without departing from the spirit and scope of the invention.

We claim:

1. An osteoinductive bone graft substitute composition comprising, in combination: about 86-89% by weight of a calcium phosphate particulate mineral component and about 11-14% by weight of a purified fibrillar collagen, said mineral component including about 20% to about 60% by weight of hydroxyapatite and about 80% to about 40% by weight of tricalcium phosphate, wherein said composition does not return to its original shape upon hydration or manipulation.

2. The composition according to claim 1 wherein said tricalcium phosphate substantially comprises beta tricalcium phosphate and said collagen substantially comprises Type I non-reconstituted bovine collagen.

3. The composition according to claim 2 comprising about 87.5% by weight of calcium phosphate particulate mineral component and about 12.5% by weight of purified fibrillar collagen, wherein said mineral component comprising about 30% to about 50% by weight hydroxyapatite and about 70% to about 50% by weight tricalcium phosphate.

4. The composition according to claim 2 comprising about 87.5% by weight calcium phosphate particulate mineral component and about 12.5% by weight purified fibrillar collagen, wherein said mineral component comprising about 50% by weight hydroxyapatite and about 50% by weight tricalcium phosphate.

5. The composition according to claim 2 wherein said calcium phosphate particulate mineral component comprise irregularly-shaped granules having a diameter of about 0.5 mm to about 1 mm.

6. The composition according to claim 1 configured as at least one of a strip.

7. The composition according to claim 2 configured as at least one of a strip having a structure including one or more interconnected pores, wherein said one or more pores have an average pore size of about 100 mm to about 800 mm.

8. The composition according to claim 7 wherein said at least one of a strip have a thickness of about 1 mm to about 5 mm.

9. The composition according to claim 7 wherein said at least one of a strip have a thickness of about 3 mm.

10. The composition according to claim 7 wherein said at least one of a strip have a width of about 3 mm to about 100 mm.

11. The composition according to claim 7 wherein said at least one of a strip have a length of about 3 mm to about 100 mm.

12. The composition according to claim 7 further comprising an effective amount of one or more compounds selected from the group consisting of autogenous bone marrow, transforming growth factor-beta (TGF-beta}, platelet-derived growth factor (PDGF), fibroblast growth factor (FGF), bone morphogenetic protein (BMP), hyaluronic acid, thermoplastic lactides and antibiotics, said one or more compounds configured to promote regrowth of bone.

13. An osteoinductive bone graft substitute composition comprising, in combination: about 86-89% by weight of a ceramic component and about 11-14% by weight of a purified fibrillar collagen, wherein said composition does not return to its original shape upon hydration or manipulation.

14. A package configured to store a bone graft composition comprising, in combination: at least one of a strip of said bone graft composition including about 86-89% by weight of a calcium phosphate particulate mineral component and about 11-14% by weight of purified fibrillar collagen, said mineral component including about 20% to about 60% by weight hydroxyapatite and about 60% to about 20% by weight beta-tricalcium phosphate, wherein said composition does not return to its original shape upon hydration or manipulation; and
said package comprises an inner sterile polymeric V-shaped pouch located in an outer sterile polymeric V-shaped pouch.

15. A method for repairing a bone defect in a patient comprising the steps of:
providing at least one of a strip and a block of an osteoconductive bone graft composition comprising about 86-89% by weight of a calcium phosphate particulate mineral component and about 11-14% by weight of purified fibrillar collagen substantially comprising Type I collagen, said mineral component including about 20% to about 60% by weight hydroxyapatite and about 60% to about 20% by weight beta-tricalcium phosphate, wherein said composition does not return to its original shape upon hydration or manipulation;
placing said patient in an aseptic operating room;
opening a wound to access said bone defect;
filling said bone defect with at least one of said strip and said block; and
closing said wound.

16. The method of claim 15 wherein said bone defect has a volume of no greater than about 30 ml.

17. The method of claim 15 further comprising the steps of:
debriding and managing said wound associated with said bone defect prior to filling said bone defect, wherein periosteal stripping is minimized;
treating a contaminated portion of said wound with at least one prophylactic antibiotic;
collecting autogenous bone marrow from at least one of an iliac crest and a fracture site of an uncontaminated wound while avoiding blood collection with said bone marrow;
selecting at least one of said strip and said block sized to fit into said bone defect;
transferring at least one of said strip and said block to a sterile tray;
adding a sterile saline solution to said sterile tray;
hydrating at least one of said strip and said block for a period of about one to three minutes;
providing another sterile tray including said bone marrow;
transferring said at least one of said strip and said block to said another sterile tray;
coating at least a portion of the surface of said at least one of said strip with said bone marrow; and
placing said at least one bone graft composition into said bone defect so that molding is minimized, wherein if molding is required bone marrow cells of said bone marrow are substantially undamaged.

18. The method of claim 17 wherein said at least one of said strip and said block at least one of said strip and is about 15 mm wide, about 45 mm long, and about 3 mm thick, and bone marrow is coated in an amount of about 5 ml for each of said strips.

19. The method of claim 15 further comprising the step of inserting an internal fixation device prior to selecting at least one of said strip and said block sized to fit into said bone defect.

20. The method of claim 15 further comprising the step of inserting an external fixation device after closure of said wound.

21. The method of claim 15 further comprising the step of surgically creating said bone defect.

21. The method of claim 15 wherein said bone defect results from bone trauma.
